(19) United States
(12) Patent Application Publication
(10) Pub. No.: US 2005/0031498 A1
(43) Pub. Date: Feb. 10, 2005

(54) Air freshener with container

(76) Inventor: Robert F. Held, Defiance, MO (US)

Correspondence Address: Paul M. Denk, 763 S. New Ballas Road, St. Louis, MO, 63141, US

(21) Appl. No.: 10/459,839

(22) Filed: Jun. 12, 2003

Related U.S. Application Data
(60) Provisional application No. 60/390,322, filed on Jun. 24, 2002.

(51) Int. Cl. A61L 9/12

(52) U.S. Cl. 422/124

(57) ABSTRACT

A containerized air freshener, having a formulation of various chemical ingredients that provide a heavily viscous or paraffin-like material that, as it evaporates or dissipates, as a result of air passing around its container, disseminates an aroma into the stream of passing air, thereby adding to the pleasant scent of every room in the house, and eliminating any unwanted odors. The container contains a front wall, back wall, and a peripheral inclined outer edge, which accommodates the flow of air there passed, and enhances the generation of reduced pressure, or slight suction, at the surface of the formulation, to provide for its attraction into the passing air. A seal may be provided upon the wall of the container, to seal the formulation in place, once it has been filled within the container, with the seal being removed, once the containerized air freshener has been fastened to a cold air return vent or register by means of a magnet, or a clip, to hold the same in place during usage.

### CROSS REFERENCE TO RELATED APPLICATION

This application is a non provisional patent application based on the provisional patent application having Ser. No. 60/390,322, which was filed on Jun. 24, 2002.

### BACKGROUND OF THE INVENTION

There are a variety of devices available upon the market for use for refreshing air within a home, business, or the like. For example, there are air fresheners that may be suspended in the home, and simply through its natural evaporation, provide for the emission of a scent into the ambient air. There are other types of air fresheners that are treated, or contain as an ingredient, an aroma, whether it be of a perfume, or the like, and which when provided in the home, furnishes an emission of its scent throughout the surrounding region. In addition, there are even air fresheners that may be used in conjunction with, for example, a night light, and which apparently when subject to some heat, such as from the night light, emits a scent into the surrounding atmosphere. These are examples of the type of air fresheners that are available in the market. There are others that incorporate a wick, that extends down into a scented liquid, and when the container is opened, and the wick pulled partially free, emits a pleasing scent.

### SUMMARY OF THE INVENTION

This invention relates generally to an air freshener, and more specifically to a packaged air freshener which may be appended or affixed directly to the outside of the cold air return vent of a household air conditioning or heating system, so as to circulate its fragrance throughout the residence, building, or the like.

This invention contemplates the formation of a container, which may be of a size that can be appended, either by clipping, magnetic attachment, or other fastening means, to an air return vent for a residential ductwork of a heating and air conditioning system. Or, it can likewise even be used within a business establishment, or the like. Essentially, the container is of a size to accommodate the filling with an ample capacity of the fragrance composition, and which, over time, due to its exposure to the moving air, will gradually release its fragrance into the moving air, as it passes through the vent, primarily the air vent, of a conditioning system.

The container is of a size that normally includes a frontal portion that is larger, in diameter, or dimension, than the back of the system, so that the moving air may pass around the containerized formulation, and provide a negative pressure exposure of the packaged fragrance, to the moving air, during its application.

The containerized formulation is located within its interior of the container, and its enclosed therein, once packaged, through the use of a covering seal, made of paper, polymer, or the like, and which may have a pressure sensitive adhesive around its perimeter, for a more fixed attachment to the container opening, so that once the seal is peeled back, its internally contained formulation becomes exposed to the passing air, and can release its fragrance and scent, into the air, without any further participation on the part of the installer.

The formulation, preferred for this application, may be made up of a combination fragrance, and or odor neutralizer. The ingredients may be included in those quantities normally used to form a composition of this type, and due to select of its ingredients, gradually emits its fragrance into the passing air, to imbue it with a pleasing scent, depending upon the type of fragrance added into the formulation. Obviously, the fragrance may be of any type, whether it be of a pine scent, a fruit scent, such as peach, or any other type of scent, or which may even include a perfume type of aroma, when it dissipates its scent into the surrounding air, particularly as it passes through the register or vent of the conditioning system.

FORMULATION

| INGREDIENT | PERCENT |
|---|---|
| Propylene Glycol | 50-70% |
| Water | 5-15% |
| Fragrance | 5-15% |
| Pantenol | 1-3% |
| PVP | 1-3% |
| Glycerin | 3-6% |
| Propylparaben | 3-5% |

Obviously, these percentages may be varied, depending upon the setting upon which the air freshener is to be used.

One of the primary objects of this invention is to provide for an air freshener that may be readily applied to a cold air return vent location within the home.

Another object of this invention is to provide for a facile and effective way to protect the home from unwanted odors.

Another object of this invention, due to the formulation of ingredients contained within this air freshener, is to provide a freshener that keeps the building or residence fresh for weeks at a time.

A further object of this invention is to provide for a container for formulation of air freshener that may easily attach to any wall of an air return vent, and works most effectively every time the heating and cooling system is initiated.

A further object of this invention is to provide for a formulation of ingredients for an air freshener that includes natural organic fragrance that actually neutralizes any unwanted odors in the home, but at the same time, being nontoxic and biodegradable.

Still another object of this invention is to provide for a very light scent of air freshener that will work continuously to eliminate odors within the home.

These and other objects may become more apparent to those skilled in the art upon reviewing the summary of the invention as provided herein, and upon undertaking a study of the description of its preferred embodiment, in view of the drawings.

### BRIEF DESCRIPTION OF THE DRAWINGS

In referring to the drawings, FIG. 1 is a front view of the container for air freshener of this invention;

FIG. 2 is a back view thereof;

FIG. 3 is a top view thereof;

FIG. 4 is a front view of the package cover;

FIG. 5 is another front view of the package cover;

FIG. 6 is a front view of a shelf tray for holding a series of the air fresheners during packaging and marketing; and

FIG. 7 is a side view of the shelf tray.

### DESCRIPTION OF THE PREFERRED EMBODIMENT

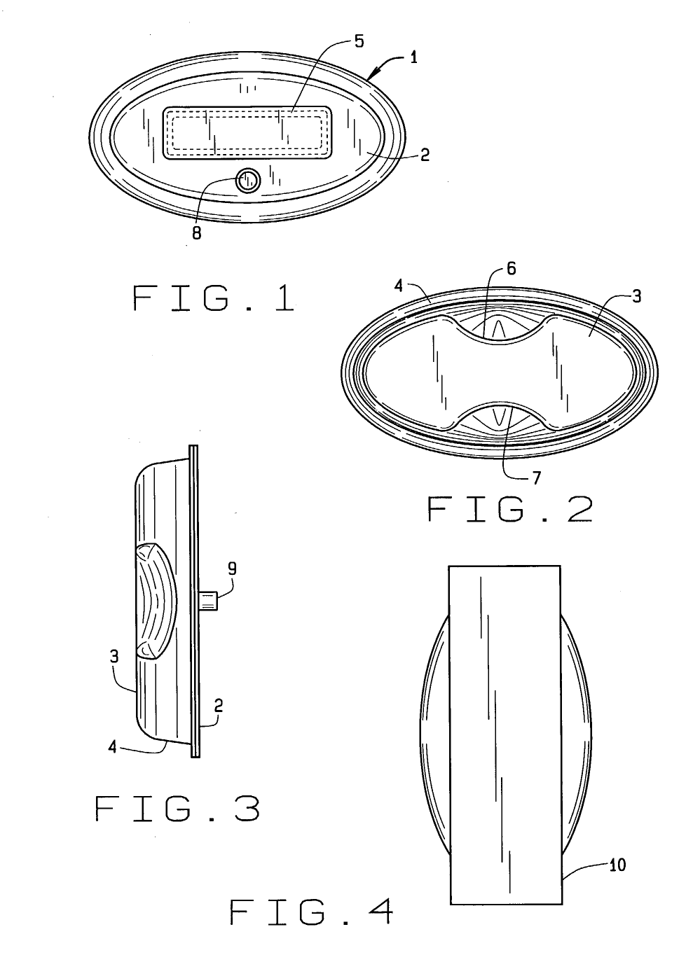

In referring to the drawings, and in particular FIGS. 1 through 3, the container for air freshener is readily disclosed. As can be noted, it has a particular shape, in this particular instance, in the form of a oval, as at 1, which is generally of a flattened surface, on the front side, as at 2, while the backside, as at 3, is likewise of an oval shaped flattened surface, intervened by an inclined outer peripheral edge wall 4, as noted. Generally the purpose for the bevel around the perimeter of the container is to provide for uniform air flow, around the container, particularly of a reducing dimension, so as to develop somewhat of a lower pressure, on its backside, in the form of a slight suction, where this container for air freshener is applied to the surface of a register, vent, or the like, for an air return for a heating and air conditioning system within a home, commercial building, or the like. Thus, when the seal 5 is removed from the front surface of the container, and exposes the type of air freshener formula, as previously summarized herein, and contained within the container, to the atmospheric air, the air passing over the front of the container, around its canted perimeter edge 4, and into the register, attracts the scent, allowing it to permeate the air, such that when the air flow is subsequently cooled, or heated, and then discharged back into the room, through a register, it has a pleasing aroma, of the flavor or type for the formula used, that is emitted back into the room, and elsewhere in the building, during usage of this device. As previously summarized, the seal may be of a paper or polymer, having a pressure sensitive adhesive, at least around its perimeter, so as to seal against the front surface or wall 2 of the container, when manufactured.

As can also be noted from this invention, the back wall 3 of the container may form a pair of indentations, as at 6 and 7, and into which fingers may insert, to provide for grasping of the container, as when it is desired to peel the seal 5 from its front surface, in preparation for its application. Also, provided upon the container, but usually upon its back wall, is a magnet, as at 8, and which may be used for applying the container to the front of the vent, particularly where the vent may be fabricated of metal, as many of them are. Or, a clip, as 9, may be furnished upon the upper edge of the device, and allow for fastening of the air freshener container directly to the vent or register, during its installation, particularly where the vent may be formed of a polymer, and not adhering of a magnetic material.

Obviously, while the container of this invention is shown to be fabricated of an oval design, for pleasing purposes, the shape of the container is not essential, and it may be fabricated to a round shape, square rectangular, or to any other shape that may add to the aesthetics of the air freshener of this invention.

FIGS. 4 and 5 may show the individual package covers, as at 10, and which may provide a sleeve that may formed around the container, during individual packaging of each of the air fresheners of this invention. The front of the sleeve, or package cover, may contain identifying indicia, such as the trademark, the type of scent or aroma that may be generated from the air freshener, and other instructions that may identify how it may be used and employed.

With respect to the implementation of this device, the purchaser may remove the outer wrap or liner 10 for the individual container. Then, the flat back side of the container, bearing the magnet, or the extending clip, may be applied directly to the vent or register. Generally, this will be applied to the air return vent, as previously explained. On the other hand, while it may not be as effective, it could be applied also to the vent that discharges the treated air back into the room, as can be readily understood. Just prior to then, this seal will have been removed from the front of the container, so as to expose its formulation to the passing air, and allow for its evaporation or discharge of the aroma and fragrance into the surrounding air, during usage. The magnet may either hold the container directly to a metallized vent, or the clip may slide between the register, and the wall opening, in order to hold the container in place.

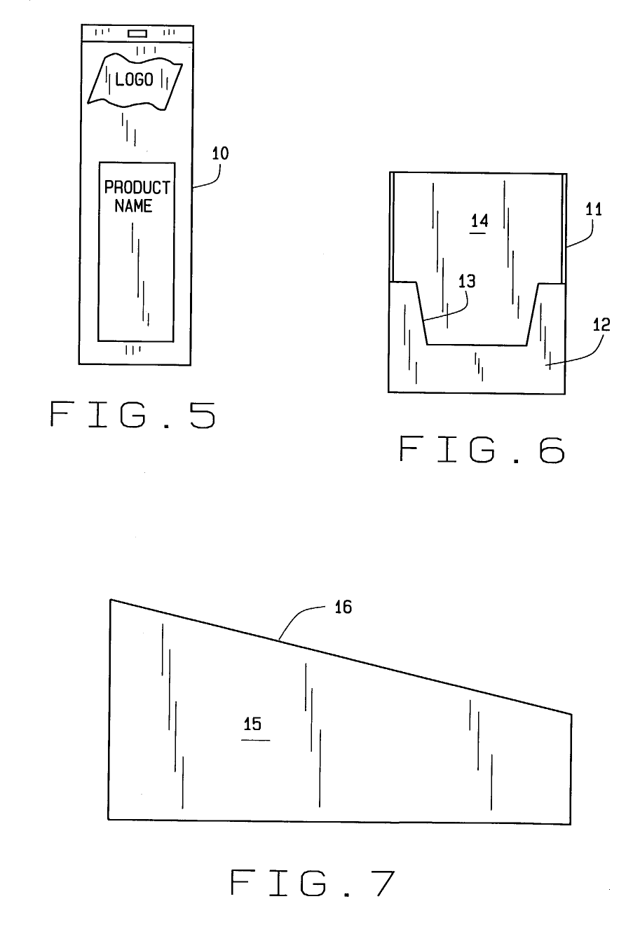

FIGS. 6 and 7 show a style of shelf tray, that can be used for holding a plurality of the air freshener containers, such as eight in number, during their display and marketing. The tray 11 includes a front wall 12, having a cut-out portion 13, and a back wall 14, intervened by side walls 15 having an upper inclined edge 16 as can be noted. Thus, a multitude of these wrapped container air fresheners may be embodied in the tray, as during storage, shipment, and when located within the store, may be readily displayed as a shelf type of tray showing the type of fragrance provided, and preferably, the type of aroma and fragrance may show through the opening 13, for viewing by the prospective customer.

In usage, the container air freshener of this invention, when applied, greatly enhances the fragrance of the room in which it locates, and will help to eliminate unpleasant or stale household odors, for several weeks, while the formulation within the container lasts, and before it is entirely evaporated, or dissipated into the ambient air.

Variations or modifications to the subject matter of this invention may occur to those skilled in the art upon review of the development as described herein. Such variations, if within the spirit of this development, are intended to be encompassed within the scope of the invention of this disclosure. The description of the preferred embodiment, as set forth herein, is provided for illustrative purposes only.

1. An air freshener for use for application to a cold air return duct of a home heating/cooling installation, comprising a container, said container having a front side and a back side, the front side of said container having an opening provided therein and into which an air freshener composition may be applied, a seal normally covering said opening, but when said seal is removed, providing for the dissemination of the air freshener to the surrounding area and for its drawings into its cold air return duct, attachment means provided upon one of the front and back side of said container, the attachment means providing for connection of the air freshener container to the cold air vent, and said air freshener having a formula including a fragrance, and odor neutralizer, and a carrier for the said composition.

2. The air freshener with container of claim 1 wherein the composition for the air freshener include propylene glycol, water, fragrance, pantenol, PVP, glycerin, and propylparaben.

3. The air freshener with container of claim 2 wherein the ingredients for the formulation of the air freshener include the following percentages by weight;

| INGREDIENT | PERCENT |
|---|---|
| Propylene Glycol | 50-70% |
| Water | 5-15% |
| Fragrance | 5-15% |
| Pantenol | 1-3% |
| PVP | 1-3% |
| Glycerin | 3-6% |
| Propylparaben | 3-5% |

4. The air freshener with container of claim 2 wherein the front seal capable of peeling free from the front side of the container.

5. The air freshener with container of claim 2 wherein the adherence of the container to the vent includes an adhesive.

6. The air freshener with container of claim 5 wherein the adhesive includes a pressure sensitive adhesive.

7. The air freshener with container of claim 2 wherein the means for attaching the container to the vent comprises a magnet.

8. The air freshener with container of claim 2 wherein the container is oval shaped, the container having side edges intermediate the front side and back side of the container, and the side edges being inclined.

9. The air freshener with container of claim 8 and including finger grips provided upon the back side of the container to facilitate its grasping.

10. The air freshener with container of claim 2 wherein the means for adhering the container to the vent includes a clip.
